(19) United States
(12) Patent Application Publication
(10) Pub. No.: US 2025/0017668 A1
(43) Pub. Date: Jan. 16, 2025

(54) System and Method for Medical Procedure Optimization

(71) Applicant: EXPANDED EXISTENCE, INC., Windermere, FL (US)

(72) Inventors: ROBERT L. MASSON, WINDERMERE, FL (US); RAYMOND G. OKTAVEC, FORT LAUDERDALE, FL (US); Kyle A. Masson, Windermere, FL (US); Brett Luc Masson, Windermere, FL (US)

(21) Appl. No.: 18/352,122

(22) Filed: Jul. 13, 2023

(51) Int. Cl. A61B 34/00; A61B 34/20; A61B 90/00

(52) U.S. Cl. CPC A61B 34/25; A61B 34/20; A61B 90/36; A61B 2034/2055; A61B 2034/254; A61B 2034/256; A61B 2090/365

(57) ABSTRACT

A system for optimizing a medical procedure in a medical procedure room is described. The server is configured to obtain data associated with medical items present in a predefined area of the medical procedure room. The server identifies an upcoming phase of the medical procedure. Further, the server obtains reference data for a plurality of reference medical items required for the upcoming phase of the medical procedure from reference medical procedures performed prior to the medical procedure. The server further compares the reference data for the plurality of reference medical items with the received data associated with the medical items present in the medical procedure room and provides recommendations for optimizing the medical procedure based on the comparison.

### BACKGROUND OF THE INVENTION

According to data from the United States Surgical Procedures Market Report 2022, over one hundred (100) million surgical procedures were performed in 2022 in the United States alone, and that number continues to grow. The setup and usage of medical items in the medical procedure room are the most important factors for medical practitioner efficiency, patient safety, and team workflow. The setup and usage of the medical items in the medical procedure room should be optimized for efficiency and procedure predictability.

Before a medical procedure begins, the medical practitioner's preferred technique related to the usage of the medical items, as well as the medical procedure room setup, must be carefully planned. Moreover, each phase of the medical procedure has different requirements in terms of the usage and setup of the medical items. The medical practitioners must be able to quickly and accurately determine whether all required medical items have been placed in the medical procedure room, is in the right location, and that the provided medical item is of the correct type, size, and/or number to suit the patient and ensure the medical procedure is performed safely and correctly. The medical procedure room must be equipped with an adequate number, and the right type, of supplies and tools, such as surgical instruments, lights, trays, robotic systems, anesthetic systems, scalpels and blades, and reusable and disposable supplies. Not only must the fixed or semi-fixed equipment be properly arranged prior to the commencement of the medical procedure, but chargeable supplies (for example, sutures, sponges, clips, medical implants, screws, rods, arthroplasty devices, stimulators, needles, scalpel blades, catheters, drill bits) and disposable supplies (for example, gauze, gloves, liners, needles, syringes, and tubing) should be carefully tracked for billing and supply analysis and inventory management. During a medical procedure that demands the full attention of the medical practitioner and other medical personnel, the tracking of such medical items can easily be ignored or incorrectly logged.

Finally, most medical procedures demand that the steps be performed in a specific order, at a specific speed, with correctly sized implants and/or tools, and the like. Regardless of how well trained the medical practitioner is, human error can occur, especially when performing new or complex medical procedures. Therefore, a medical practitioner must pay careful attention to the sequence of medical procedure steps, the number and characteristics of all tools and supplies used, and the medical procedure room setup, to name a few.

### BRIEF DESCRIPTION OF THE SEVERAL VIEWS OF THE DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention and explain various principles and advantages of those embodiments.

FIG. 1 is a block diagram of a system for optimizing a medical procedure in a medical procedure room, in accordance with some embodiments.

FIG. 2 is a plan view of an exemplary medical procedure room, in accordance with some embodiments.

FIGS. 3A through 3B illustrate a representation of reference data from one or more reference medical procedures stored in a database, in accordance with some embodiments.

FIG. 4 is a block diagram of a server for use within the system of FIG. 1, in accordance with some embodiments.

FIG. 5 is a block diagram of a user device for use within the system of FIG. 1, in accordance with some embodiments.

FIGS. 6A through 6B depict a flow diagram of a method for optimizing the medical procedure in the medical procedure room, in accordance with some embodiments.

FIGS. 7 through 9 illustrate a first predefined area of the medical procedure room when the method for obtaining data associated with usage of a medical item in the first predefined area of the medical procedure room is implemented, in accordance with some embodiments.

FIGS. 10 through 11 illustrate a second predefined area of the medical procedure room when the method for obtaining data associated with the usage of the medical item in the second predefined area of the medical procedure room is implemented, in accordance with some embodiments.

FIG. 12 illustrates the user device displaying recommendations for optimizing the medical procedure, in accordance with some embodiments.

FIG. 13 is a block diagram of an optimization cycle for use within the server, in accordance with some embodiments.

Skilled artisans will appreciate that the elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION OF THE INVENTION

In one aspect, a system for optimizing a medical procedure in a medical procedure room is described. The system includes an imaging device, a server communicatively coupled to the imaging device, and an augmented reality device communicatively coupled to the server. The imaging device is configured to obtain one or more data associated with one or more medical items present in a predefined area of the medical procedure room. The server is further configured to receive the one or more data associated with the one or more medical items present in the predefined area of the medical procedure room from the imaging device. Further, the server is configured to identify an upcoming phase of the medical procedure. Each medical procedure is divided into a plurality of phases and each phase is uniquely identified by a phase identifier. The server is further configured to obtain one or more reference data for a plurality of reference medical items required for the upcoming phase of the medical procedure from one or more reference medical procedures performed prior to the medical procedure. The one or more reference data for the plurality of reference medical items is associated with a phase identifier of the upcoming phase. The server is further configured to compare the one or more reference data for the plurality of reference medical items with the one or more received data associated with the one or more medical items present in the medical procedure room. The server is further configured to provide one or more recommendations for optimizing the medical procedure based on the comparison. The augmented reality device is configured to receive the one or more recommendations from the server and display the one or more recommendations on the augmented reality device for the optimization of the medical procedure.

In another aspect, a method for optimizing a medical procedure in a medical procedure room is described. The method includes obtaining, by at least one imaging device, one or more data associated with one or more medical items present in a predefined area of the medical procedure room. The method further includes transmitting, by the at least one imaging device, the one or more data associated with the one or more medical items present in the predefined area of the medical procedure room. Further, the method includes receiving, by the server, the one or more data associated with one or more medical items present in the predefined area of the medical procedure room from the at least one imaging device. The method further includes, identifying, by a server, an upcoming phase of the medical procedure. Each medical procedure is divided into a plurality of phases. Each phase is uniquely identified by a phase identifier. The method includes obtaining, by the server, one or more reference data for a plurality of reference medical items required for the upcoming phase of the medical procedure from one or more reference medical procedures performed prior to the medical procedure. The one or more reference data for the plurality of reference medical items is associated with a phase identifier of the upcoming phase. Further, the method includes comparing, by the server, the one or more reference data for the plurality of reference medical items with the received one or more data associated with the one or more medical items present in the medical procedure room. The method includes providing, by the server, one or more recommendations for optimizing the medical procedure based on the comparison. Further, the method includes receiving, by an augmented reality device, the one or more recommendations from the server. The method further includes displaying, on the augmented reality device, the one or more recommendations for the optimization of the medical procedure.

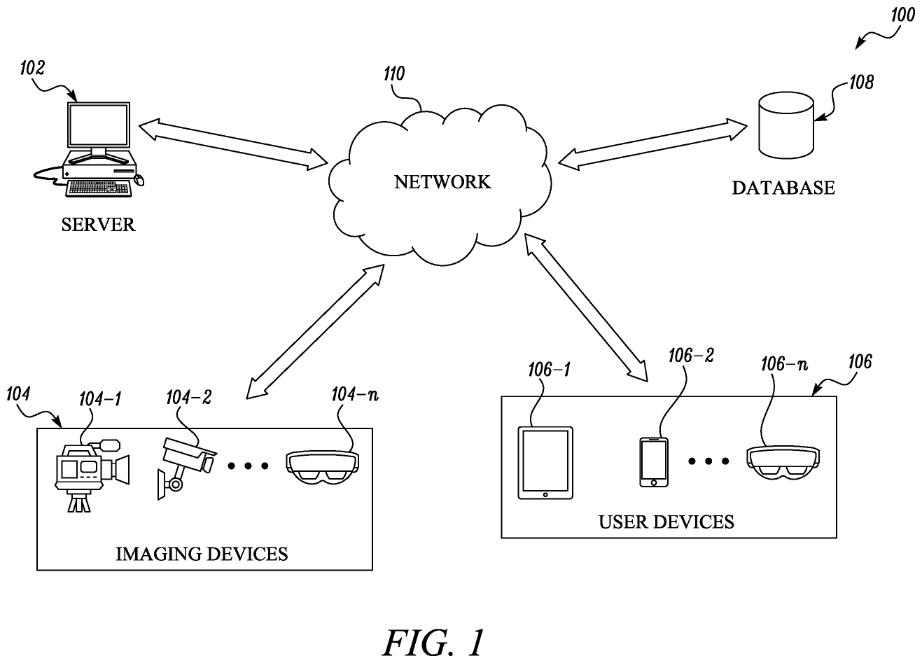

FIG. 1 is a block diagram of a system 100 for optimizing a medical procedure in a medical procedure room in accordance with various embodiments. The medical procedure corresponds to a plurality of steps that are to be carried out by medical practitioners or medical professionals to manage a medical condition in the medical procedure room. For example, the medical procedure includes, but is not limited to, a surgical procedure, a diagnostic procedure, a therapeutic procedure, and any other medical procedure now known or in the future developed. In accordance with various embodiments, optimizing the medical procedure includes optimizing a setup and/or usage and/or count of medical items in the medical procedure room for improving the accuracy and efficiency of the medical procedure.

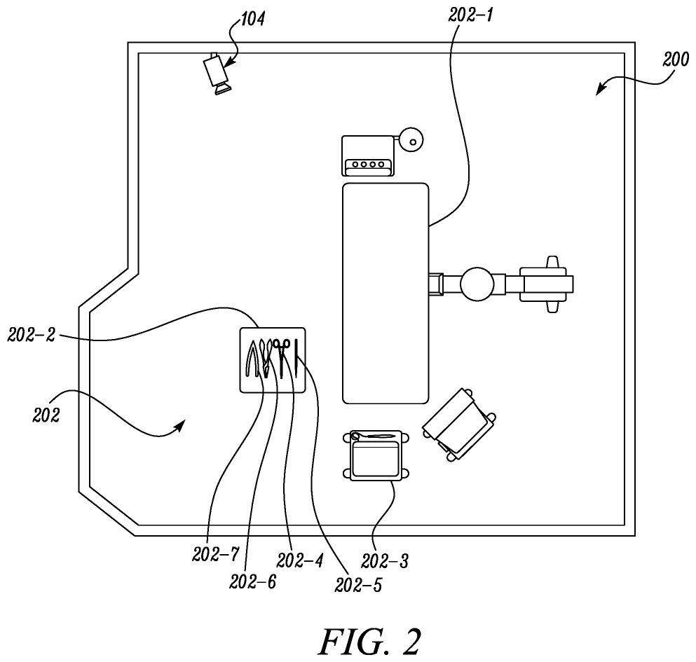

The medical procedure room is a room used for performing one or more medical procedures and includes one or more medical items for use during the medical procedures. For example, as shown in FIG. 2, a medical procedure room 200 includes the medical items 202, such as, but not limited to, a medical procedure table 202-1, one or more auxiliary tables 202-2 or stands (such as a Mayo stand), an enabling technology 202-3, a surgical scissor 202-4, a surgical needle 202-5, a forceps 202-6, a surgical tweezer 202-7, and various other chargeable and/or disposable medical items (for example, but not limited to, sutures, sponges, clips, medical implants, screws, rods, arthroplasty devices, stimulators, needles, scalpel blades, catheters, drill bits, gauzes, gloves, liners, needles, and syringes) (not shown) now known or in the future developed. It will be understood that the medical procedure room 200 is not limited to the medical items 202 shown in FIG. 2 and includes any number of items, such as, a back table, one or more storage closets, nurse workstations, back tables, anesthesia systems, electrocautery systems, enabling technology systems or workstations (for example, but not limited to, microscopes, robotic surgical systems, networked robotics, illumination systems, or any other enabling technology systems now known or in the future developed, and accompanying monitors or displays), biometric readers, and/or wireless transceivers.

The setup of a medical item 202 corresponds to an arrangement including, but not limited to, a location, a position, an orientation, and an order of placement of the medical item 202 in the medical procedure room 200. The usage of the medical item 202 corresponds to a manner in which the medical item 202 is used in the medical procedure. For example, the usage includes an order of use of the medical item 202, a time stamp associated with usage of the medical item 202, and a duration of usage of the medical item 202. The count of the medical item 202 corresponds to a number of units of a medical item 202 used/consumed during the medical procedure. The setup and/or the usage and/or the count of the medical items 202 depend on various factors such as, but not limited to, a preference of a medical practitioner, a layout of the medical procedure room 200, and a type of the medical procedure.

The system 100 provides for the optimization of the setup and/or usage and/or count of the medical items 202 in the medical procedure room 200 based on a phase (for example, a stage) of the medical procedure. To this end, each medical procedure is divided into a plurality of phases that are to be completed in a sequential manner and the plurality of steps of the medical procedure are divided into one or more sets of steps. Each set of steps is then associated with a unique phase identifier identifying a unique phase of the medical procedure. For example, a first phase of the medical procedure is a preoperative phase corresponding to a set of steps including setup and/or usage and/or count of one or more medical items, such as, a blood pressure monitor or sphygmomanometer to check the blood pressure, a glucometer to check the blood sugar levels, a temperature monitor to measure the body temperature, and an anesthesia machine for anesthesia.

In accordance with various embodiments, the optimization of the step-up and/or usage and/or count of the medical items 202 in the medical procedure room 200 is performed for each phase depending upon reference data associated with the setup and/or usage and/or count requirements of the medical items for the corresponding phase. Referring to FIG. 1, the system 100 includes a server 102, one or more imaging devices 104 (for example, but not limited to, imaging devices 104-1, 104-2, . . . 104-n), one or more user devices 106 (for example, but not limited to, user devices 106-1, 106-2, . . . 106-n), and a database 108. In some embodiments, communication between the server 102, the one or more imaging devices 104, the one or more user devices 106, and the database 108 occurs through a network 110. In some embodiments, the network 110 is, for example, a wide area network (WAN) (for example, a transport control protocol/internet protocol (TCP/IP) based network), a cellular network, or a local area network (LAN) employing any of a variety of communications protocols as is well known in the art or developed in the future.

The components and the functionality of the one or more imaging devices 104 are now described in detail. For ease of reference, the components, and the functionality of one imaging device 104 is described hereinafter, however each of the same components and functionality are applicable to other imaging devices 104 shown in FIG. 1. In accordance with various embodiments, the imaging device 104 is configured to obtain one or more data associated with the medical items 202 present in the medical procedure room 200. For example, the data includes data related to the usage and/or setup and/or count of the medical items 202 in the medical procedure room 200. The imaging device 104 is any device capable of capturing images and/or recording one or more videos of the medical items 202 in the medical procedure room 200. For example, the imaging device 104 includes one or more of a low-angle camera 104-1, a wall-mounted camera 104-2, an augmented reality device 104-n, or any device now known or in the future developed which includes a lens for capturing images and/or recording videos of the medical items 202 in the medical procedure room 200. In accordance with various embodiments, the imaging device 104 includes an image recognition software to identify and track the data associated with the medical items 202 present in the medical procedure room 200 based on the captured images and/or recorded videos. The imaging device 104 obtains the data associated with the medical items 202 and transmits the data to the server 102. Although reference has been made to the imaging device 104 to obtain the data associated with the medical items 202 present in the medical procedure room 200, it will be appreciated that any other device, such as, a Light Detection and Ranging (LiDAR) sensor, a Radio Detection and Ranging (RADAR), an ultrasonic sensor, a three-dimensional (3D) scanner, a IoT (Internet of Things) sensor, a RFID (radio frequency identification) sensor, a Quick Response (QR) code reader, a biometric sensor, and various other sensors known in the art or developed in the future may be deployed to obtain the data associated with the medical items 202.

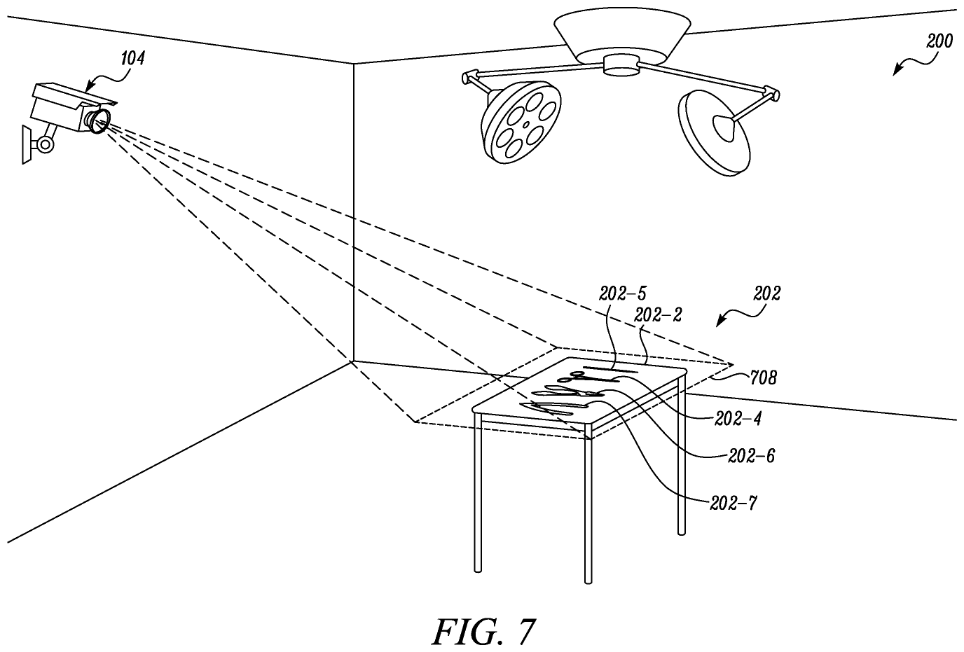

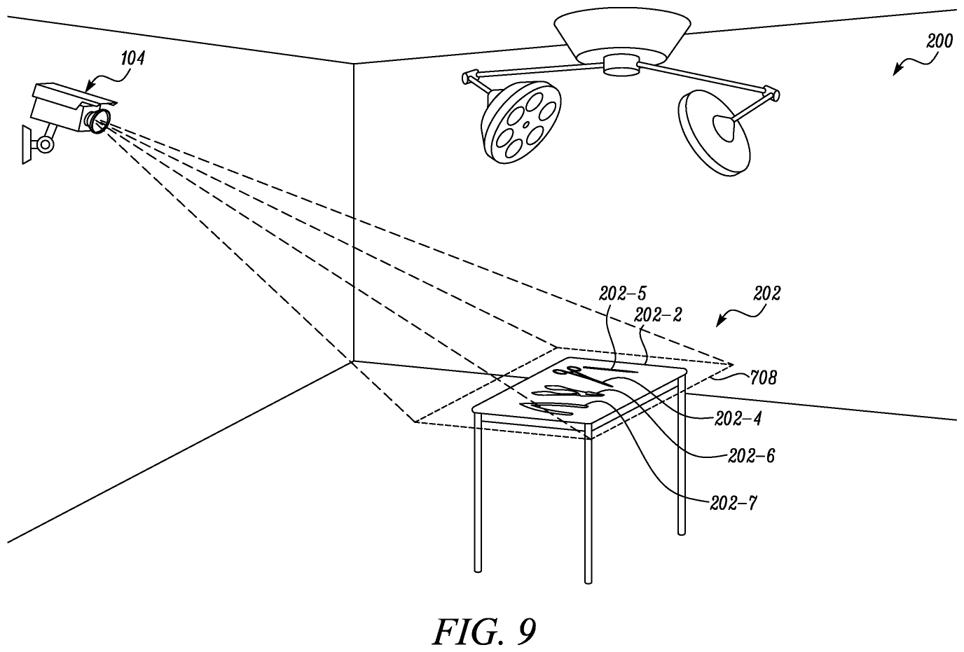

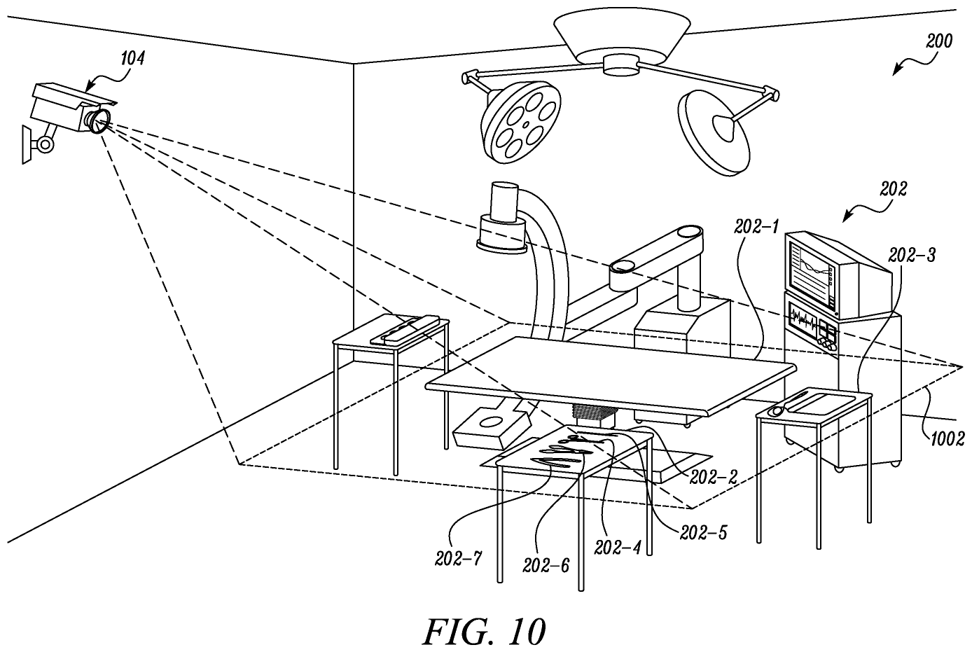

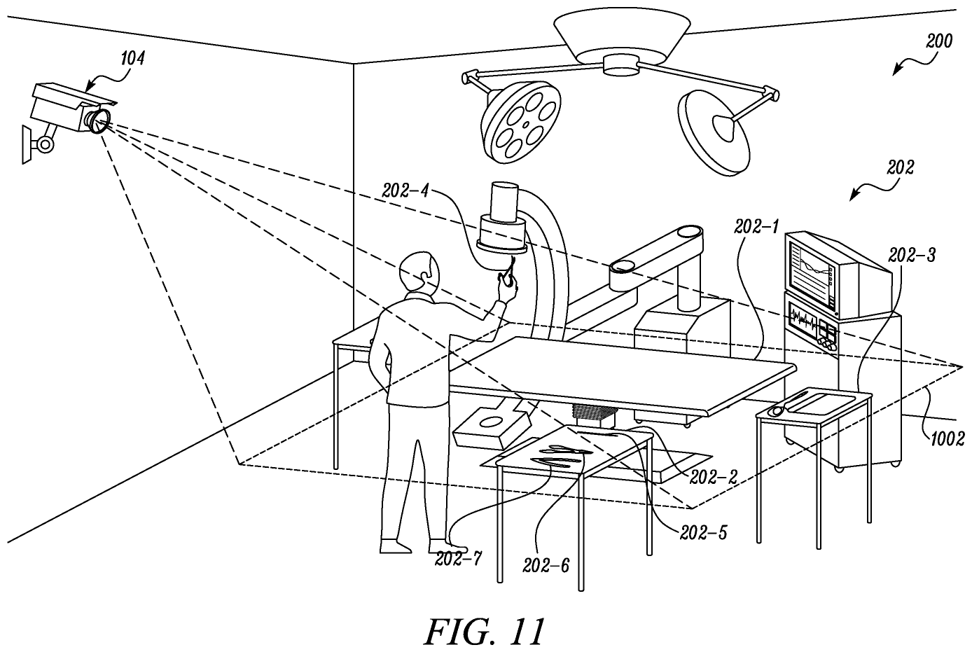

In accordance with various embodiments, the imaging device 104 is configured to obtain data associated with the medical items 202 present in a predefined area of the medical procedure room 200. The predefined area of the medical procedure room 200 includes an entire area of the medical procedure room 200 or a portion thereof. The predefined area is defined by the medical practitioner or a medical procedure room personnel based on one or more parameters, such as, but not limited to, the preference of the medical practitioner, the type of the medical procedure, and the layout of the medical procedure room 200. For example, as shown in FIGS. 7 through 9, the predefined area corresponds to a first predefined area 708 including a portion of the area of the medical procedure room 200 surrounding the auxiliary table 202-2, the surgical scissor 202-4, the surgical needle 202-5, the forceps 202-6, and the surgical tweezer 202-7. In another embodiment shown in FIGS. 10 through 11, the predefined area corresponds to a second predefined area 1002 including the entire area of the medical procedure room 200 covering all medical items 202 present in the medical procedure room 200.

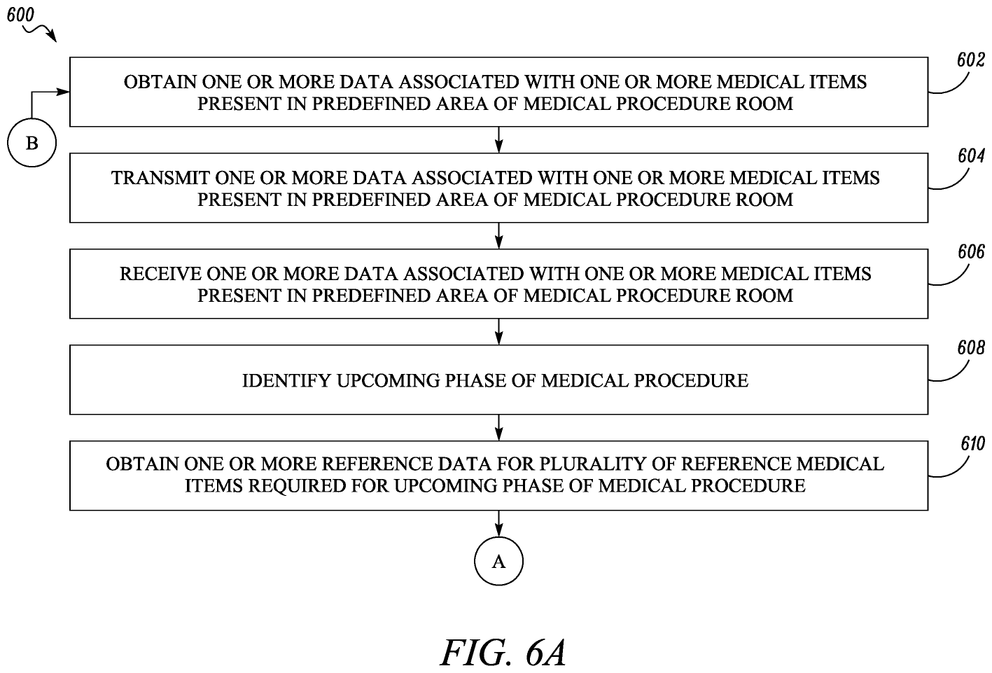

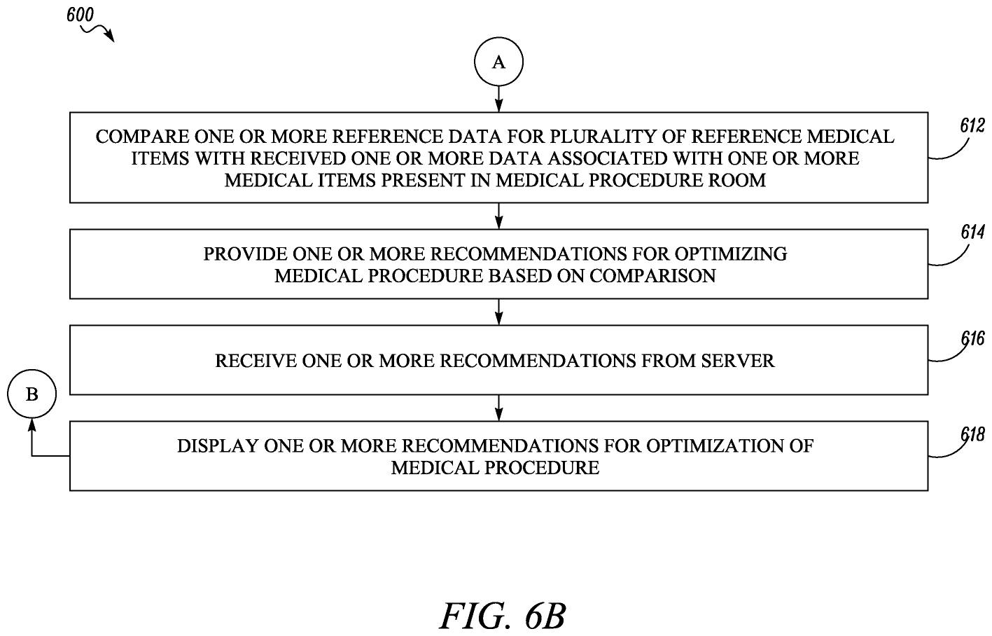

In an embodiment, the imaging device 104 is also configured to obtain data associated with the count of one or more chargeable and/or disposable medical items 202 included in the predefined area 708, 1002 of the medical procedure room 200. To this end, the imaging device 104 identifies the chargeable and/or disposable medical items 202 in the predefined area 708, 1002 of the medical procedure room 200 and tracks the number of the chargeable and/or disposable medical items utilized during each phase of the medical procedure. For example, the imaging device 104 determines, for each chargeable and/or disposable medical item 202, a number of units present in the predefined area 708, 1002 of the medical procedure room 200 at a start as well as at an end of a phase of the medical procedure. In some embodiments, the imaging device 104, determines, for each chargeable and/or disposable medical item 202, the number of units present in the predefined area 708, 1002 of the medical procedure room 200 at a start as well as at an end of the medical procedure. The imaging device 104 then provides the data to the server 102 for determining the number of the units utilized or consumed for each chargeable and/or disposable medical item during the medical procedure and/or during each phase of the medical procedure. The server 102 provides recommendations to the user device 106 to reduce wastage of the chargeable and/or disposable medical items based on the determined number, as will be described in detail with reference to FIG. 6. In some embodiments, the determined number of units is also provided to a database of an inventory management system (not shown in the figure) for inventory management and billing purposes, for example, by the server 102.

In accordance with various embodiments, the database 108 (shown in FIG. 1) includes storage of one or more data related to the setup and/or usage and/or count of a plurality of medical items, for example, the medical items 202, and various other medical items (not shown), in one or more reference medical procedures. The one or more reference medical procedures corresponds to the medical procedures performed prior to the medical procedure and the one or more reference data corresponds to data related to the setup and/or usage and/or count of the plurality of medical items 202, and the various other medical items monitored during the one or more reference medical procedures. In an embodiment, the one or more reference medical procedures include one or more first reference medical procedures for which the medical practitioner performs the medical procedure in the medical procedure room 200. In such cases, the one or more first reference medical procedures are associated with a medical practitioner identifier identifying the medical practitioner, a medical procedure identifier identifying the medical procedure, and a medical room identifier identifying the medical procedure room 200. In some other embodiments, the one or more reference medical procedures include one or more second medical procedures associated with one or more of a plurality of medical practitioner identifiers, a plurality of medical procedure identifiers, and a plurality of medical procedure room identifiers.

The one or more reference data associated with the setup and/or usage and/or count of the plurality of medical items in the one or more reference medical procedures is obtained using one or more data capturing techniques employing data capturing devices, such as, but not limited to, an imaging device (similar to the imaging device 104 described above), a user device (similar to the user device 106), a Light Detection and Ranging (LiDAR) sensor, a Radio Detection and Ranging (RADAR), an ultrasonic sensor, a three-dimensional (3D) scanner, a IoT (Internet of Things) sensor, a RFID (radio frequency identification) sensor, a Quick Response (QR) code reader, a biometric sensor, and various other sensors known in the art or developed in the future. In some embodiments, the one or more reference data or a subset of the one or more reference data is manually entered in the database 108, for example, using the server 102 and/or the user device 106.

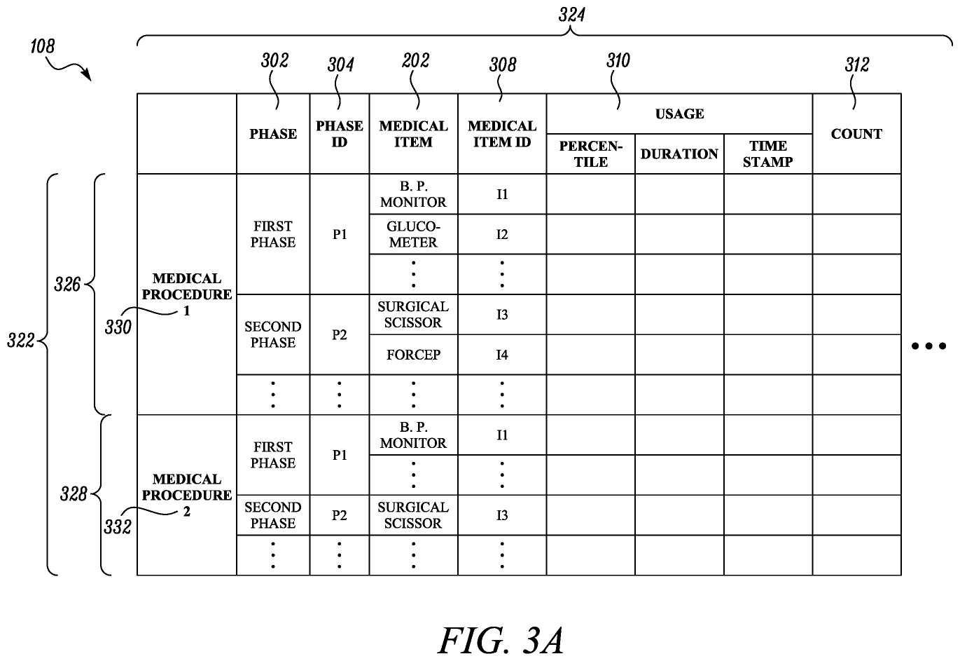

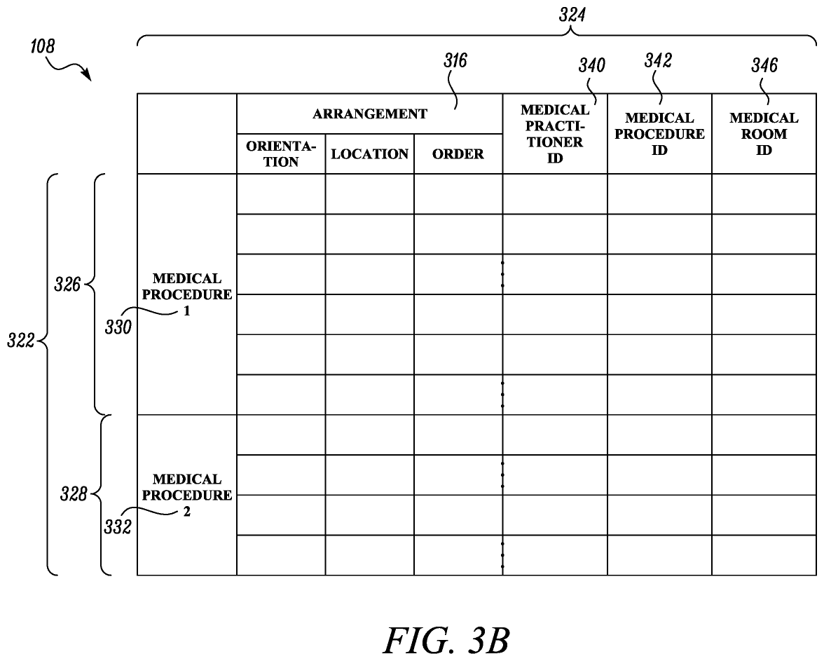

As shown in FIGS. 3A and 3B, the one or more reference data is stored in an organized manner in the database 108 having a plurality of rows 322 and columns 324. The plurality of rows 322 are divided into one or more sets of rows 326, 328 and each set of rows 326, 328 corresponds to a reference medical procedure. For example, a first set of rows 326 corresponds to a first reference medical procedure 330 and a second set of rows 328 corresponds to a second reference medical procedure 332. Each row of the first set of rows 326 and the second set of rows 328 corresponds to a unique phase (interchangeably referred to as a phase) of the corresponding reference medical procedures 330, 332, respectively. As shown in FIG. 3A, the plurality of columns 324 store data associated with a phase 302, a phase identifier 304 associated with each phase 302, and a plurality of medical items 202 associated with each phase 302 of the corresponding reference medical procedures 330, 332. As shown in FIGS. 3A and 3B, the plurality of columns 324 further includes data associated with an identifier 308, the usage 310 (including percentile, duration, time stamp of usage), the count 312 of units used/consumed for each medical item 202, and the arrangement 316 corresponding to each medical item 202. Additionally, as shown in FIG. 3B, the plurality of columns 324 includes data associated with a medical practitioner identifier 340 identifying a medical practitioner, a medical procedure identifier 342 identifying a medical procedure, and a medical procedure room identifier 346 identifying a medical procedure room associated with the corresponding reference medical procedures 330, 332.

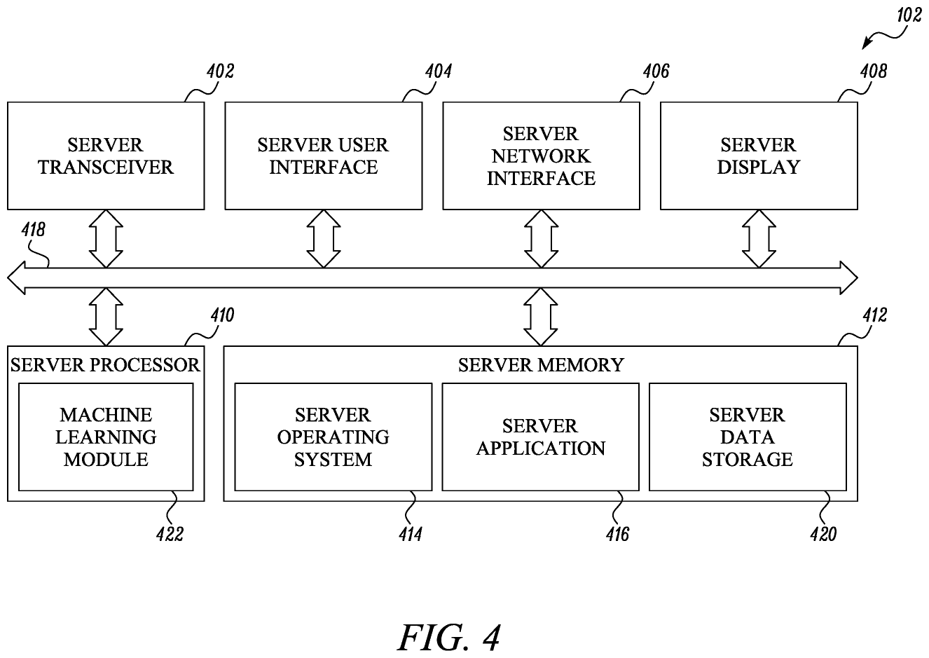

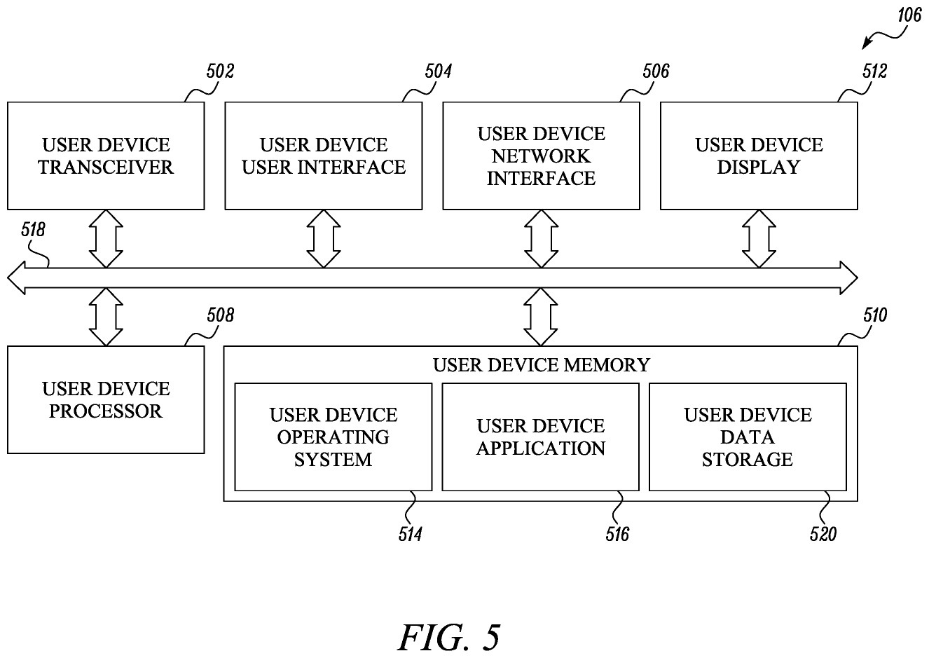

The server 102 is configured to provide one or more recommendations to the one or more user devices 106 to optimize the setup and/or usage and/or count of medical items 202 in the medical procedure room 200 based on the one or more data obtained from the imaging device 104 and the database 108. For ease of reference, the components and the functionality of the server 102 are described in detail hereinafter with reference to FIGS. 4, 6A, and 6B. In some embodiments, each of the one or more user devices 106 operates as a user interface for one or more users, such as a medical procedure room personnel or a medical practitioner, for receiving the one or more recommendations from the server 102, as will be further described with respect to FIG. 5. For ease of reference, the components, and the functionality of one user device 106 are described hereinafter, however, each of the same components and functionality are applicable to other user devices 106 shown in FIG. 1.

FIG. 4 is a block diagram of one exemplary embodiment of the server 102 for use within the system 100 of FIG. 1 in accordance with some embodiments. The server 102 is electrically and/or communicatively coupled to the imaging device 104, the user device 106, and the database 108. In some embodiments, the server 102 includes a plurality of electrical and electronic components, for example, for providing power, operational control, and communication within the server 102. For example, in one embodiment, the server 102 includes, among other things, a server transceiver 402, a server user interface 404, a server network interface 406, a server display 408, a server processor 410, and a server memory 412.

It should be appreciated by those of ordinary skill in the art that FIG. 4 depicts the server 102 in a simplified manner and a practical embodiment includes additional components and suitably configured logic to support known or conventional operating features that are not described in detail herein. It will further be appreciated by those of ordinary skill in the art that the server 102 is a personal computer, a desktop computer, a tablet, an augmented reality device, a smartphone, a wearable device (wrist worn, eye worn), or any other server now known or in the future developed. It will further be appreciated by those of ordinary skill in the art that the server 102 alternatively functions within a remote server, a cloud server, or any other remote computing mechanism now known or in the future developed.

The components of the server 102 (for example 402, 404, 406, 408, 410, and 412) are communicatively coupled via a server local interface 418. The server local interface 418 includes, for example, but not limited to, one or more buses or other wired or wireless connections, as is known in the art. In an embodiment, the server local interface 418 has additional elements, which are omitted for simplicity, such as controllers, buffers (caches), drivers, repeaters, and receivers, among many others, to enable communications. Further, in some embodiments, the server local interface 418 appropriate includes address, control, and/or data connections to enable communications among the aforementioned components.

The server processor 410 is a hardware device for executing software instructions. In an embodiment, the server processor 410 is any custom made or commercially available processor, a central processing unit (CPU), an auxiliary processor among several processors associated with the server processor 410, a semiconductor-based microprocessor, or generally any device for executing software instructions now known or in the future developed. When the server 102 is in operation, the server processor 410 is configured to execute software stored within the server memory 412, to communicate data to and from the server memory 412, and to generally control operations of the server 102 pursuant to the software instructions.

The server processor 410 includes a machine learning module 422 configured to learn and adapt itself to continuous improvement in changing environments. The machine learning module 422 employs any one or combination of the following computational techniques: neural network, constraint program, fuzzy logic, classification, conventional artificial intelligence, symbolic manipulation, fuzzy set theory, evolutionary computation, cybernetics, data mining, approximate reasoning, derivative-free optimization, decision trees, and/or soft computing. The machine learning module 422 implements an iterative learning process. The learning is based on a wide variety of learning rules or training algorithms. In an embodiment, the learning rules include one or more of back-propagation, patter-by-pattern learning, supervised learning, and/or interpolation. The machine learning module 422 is configured to implement one or more machine learning algorithms to provide the one or more recommendations to optimize the setup and/or usage and/or count of the medical items 202 of the medical procedure. In accordance with some embodiments of the invention, the machine learning algorithm utilizes any machine learning methodology, now known or in the future developed, for classification. For example, the machine learning methodology utilized includes one or a combination of: Linear Classifiers (Logistic Regression, Naive Bayes Classifier); Nearest Neighbor; Support Vector Machines; Decision Trees; Boosted Trees; Random Forest; and/or Neural Networks. The machine learning module 422 continually evolves the specifics of the setup and/or usage and/or count of the medical items 202 in the medical procedure room 200 in real time with new data inputs. The machine learning intent is to continually implement optimized setup and/or usage and/or count of the medical items 202 for performing the medical procedure in the medical procedure room 200 over time. The detailed functionalities and operations of the server processor 410 including the machine learning module 422 will be described hereinafter in greater detail.

The server user interface 404 is used to receive user input from and/or for providing system output to the user (for example, the medical procedure room personnel or the medical practitioner) or to one or more devices 106. User input is provided via, for example, a keyboard, touchpad, a mouse, and/or any other user input now known or in the future developed, or any combination thereof. System output is provided via a server display 408, speakers, a printer (not shown) and/or any other system output now known or in the future developed, or any combination thereof. The server user interface 404 further includes, for example, a serial port, a parallel port, an infrared (IR) interface, a universal serial bus (USB) interface, and/or any other interface now known or in the future developed.

The server network interface 406 is used to enable the server 102 to communicate on a network, such as, the network 110 of FIG. 1, a wireless access network (WAN), and a radio frequency (RF) network. The server network interface 406 includes, for example, an Ethernet card or adapter or a wireless local area network (WLAN) card or adapter. Additionally, or alternatively, the server network interface 406 includes a radio frequency interface for wide area communications, such as Long-Term Evolution (LTE) networks, or any other network now known or in the future developed. In an embodiment, the server network interface 406 includes address, control, and/or data connections to enable appropriate communications on the network.

The server display 408 includes a display screen or a computer monitor now known or in the future developed. In accordance with some embodiments, the server display 408 is configured to display any data, such as, the recommendations associated with the setup and/or usage and/or count of the medical items 202 in the medical procedure room 200, a list of phases of the medical procedure, inputs to modify the recommendations received from the user device 106, or the reference data from the database 108.

The server memory 412 includes any non-transitory memory elements comprising one or more of volatile memory elements (for example, random access memory (RAM), nonvolatile memory elements (for example, read-only memory “ROM”), and combinations thereof). Moreover, the server memory 412 incorporates electronic, magnetic, optical, and/or other types of storage media now known or in the future developed. Note that, in some embodiments, the server memory 412 has a distributed architecture, where various components are situated remotely from one another, but are accessed by the server processor 410. The software in the server memory 412 includes one or more software programs, each of which includes an ordered listing of executable instructions for implementing logical functions. The software in the server memory 412 includes a server operating system 414 and one or more server applications 416. The server operating system 414 controls the execution of other computer programs, such as, the one or more server applications 416, and provides scheduling, input-output control, file and data management, memory management, and communication control and related services. The one or more server applications 416 are configured to implement the various processes, algorithms, methods, techniques described herein.

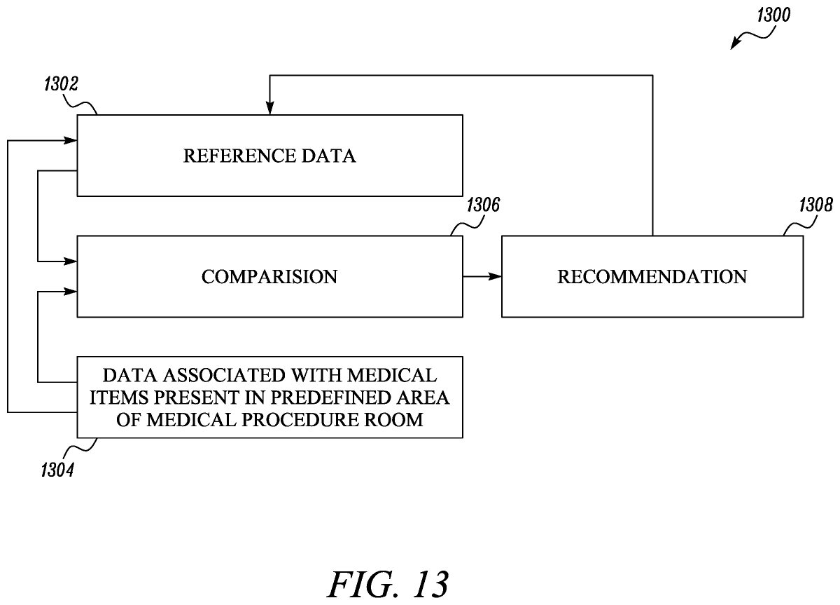

The server memory 412 further includes a server data storage 420 used to store data. In the exemplary embodiment of FIG. 4, the server data storage 420 is located internal to the server memory 412 of the server 102. Additionally, or alternatively (not shown), the server data storage 420 is located external to the server 102 such as, for example, an external hard drive connected to the server user interface 404. In some embodiments (not shown), the server data storage 420 is located external and connected to the server 102 through a network and accessed via the server network interface 406. In some embodiments, the externally located server data storage 420 corresponds to the database 108 that stores the reference data associated with the one or more reference medical procedures 330, 332 (shown in FIGS. 3A and 3B). Alternatively, in other embodiments, the server data storage 420 and the database 108 are distinct storage units. In such cases, the server data storage 420 is configured to store data, such as, the data associated with the medical items 202 obtained from the imaging device 104 and the recommendations to optimize the setup and/or usage and/or count of the medical items 202. In some embodiments, the data stored in the server data storage 420 is further provided to the database 108 during or after the execution of the medical procedure for updating the reference data stored in the database 108, as described in detail with respect to the FIG. 13.

In operation, information for storage in the server data storage 420 is entered via the server user interface 404. Alternatively, information for storage in the server data storage 420 is received from the imaging device 104 or the database 108, via the server transceiver 402. Alternatively, information for storage in the server data storage 420 is received from one or more sensors (not shown) external to the server 102 via the server transceiver 402.

The server 102 in the illustrated example includes the server transceiver 402. The server transceiver 402 incorporating within a server transceiver antenna (not shown), enables wireless communication between the server 102 and other devices, for example, the imaging device 104, the user device 106, and the database 108. It will be appreciated by those of ordinary skill in the art that the server 102 includes a single server transceiver 402 as shown, or alternatively separate transmitting and receiving components, for example, but not limited to, a transmitter, a transmitting antenna, a receiver, and a receiving antenna and/or any combination thereof.

FIG. 5 is a block diagram of one exemplary embodiment of the user device 106 for use within the system 100 of FIG. 1. In accordance with some embodiments, the user device 106 is a tablet 106-1, a smartphone 106-2, an augmented reality or wearable device 106-n, or any other computer or user device now known or in the future developed.

In an embodiment, the user device 106 being an augmented reality device 106-n provides an augmented reality interface in which a direct or indirect view of real-world environments, for example, the medical procedure room 200, in which the user of the augmented reality device 106-n is currently disposed are augmented (for example, supplemented, by additional sensory input such as sound, images, virtual representation of medical items, digital identification mark, color coding, graphics, or other information, generated by the server 102). In still other embodiments, the augmented reality device 106-n provides a mixed reality interface in which electronically generated objects, for example, virtual representations of medical items 202, color coding, and/or digital identification marks, are inserted in a direct or indirect view of real-world environments in a manner such that they co-exist and interact in real time with the real-world environment and real-world objects of the medical procedure room 200. It will be appreciated by those of ordinary skill in the art that the augmented reality device 106-n comprises any augmented reality, mixed reality, or virtual reality technology now known or in the future developed. It will further be appreciated by those of ordinary skill in the art that the augmented reality device 106 is one or more of a head-mounted display device (in the form of eyeglasses, goggles, a helmet, a visor), a handheld display device, or any other form of augmented reality device now known or in the future developed. It will further be appreciated by those of ordinary skill in the art that the augmented reality device 106-n generates and/or displays augmented reality images.

In some embodiments, the user device 106 is electrically and/or communicatively coupled to a variety of other devices, for example, the server 102, and the database 108. The user device 106 includes a number of electrical and electronic components, providing power, operational control, communication within the user device 106. For example, the user device 106 in one embodiment includes, among other elements, a user device transceiver 502, a user device user interface 504, a user device network interface 506, a user device processor 508, a user device memory 510, and a user device display 512.

The components of the user device 106 (for example 502, 504, 506, 508, 510, 512) are communicatively coupled via a user device local interface 518. The user device local interface 518 includes, for example, but is not limited to, one or more buses or other wired or wireless connections, as is known in the art. In an embodiment, the user device local interface 518 has additional elements, which are omitted for simplicity, such as controllers, buffers (caches), drivers, repeaters, and receivers, among many others, to enable communications. Further, in some embodiments, the user device local interface 518 includes address, control, and/or data connections to enable appropriate communications among the aforementioned components.

The user device processor 508 is a hardware device for executing software instructions now known or in the future developed. In an embodiment, the user device processor 508 is any custom-made or commercially available processor, a central processing unit (CPU), an auxiliary processor among several processors associated with the user device processor 508, a semiconductor-based microprocessor, or generally any device for executing software instructions. When the user device 106 is in operation, the user device processor 508 is configured to execute software stored within the user device memory 510, to communicate data to and from the user device memory 510, and to generally control operations of the user device 106 pursuant to the software instructions. The detailed functionalities and operations of the user device processor 508 will be described hereinafter in greater detail.

The user device user interface 504 is used to receive input from the server 102 and/or for providing system output to the user (for example, the medical procedure room personnel) or to one or more devices. The user device user interface 504 includes one or more input devices, including but not limited to a navigation key, a function key, a microphone, a voice recognition component, a joystick, or any other mechanism capable of receiving an input (for example, a selection of a phase of the medical procedure) from a user now known or in the future developed, or any combination thereof. Further, the user device user interface 504 includes one or more output devices, including but not limited to a speaker, headphones, a display, or any other mechanism capable of presenting an output (for example, the recommendations) to a user now known or in the future developed, or any combination thereof. In some embodiments, the user device user interface 504 includes a user interface mechanism, such as a touch interface or gesture detection mechanism that allows a user to interact with the displayed output.

As illustrated, the user device display 512 is a separate user interface or combined within the user device user interface 504. In some embodiments, the user device display 512 is configured to display a list of phases associated with the medical procedure for selection by the user. In an embodiment when the user device 106 is an augmented reality device 106-n, then the user device display 512 provides a two-dimensional or three-dimensional image, like an augmented reality view of the medical procedure room 200, visible on the user device 106. The user device display 512 is, for example, a projection device for displaying information such as text, images, graphics, or video received from the server 102 via the network 110 of FIG. 1.

The user device network interface 506 is used to enable the user device 106 to communicate on a network, such as, the network 110 of FIG. 1, a wireless access network (WAN), and a radio frequency (RF) network. In an embodiment, the user device network interface 506 includes, for example, an Ethernet card or adapter or a wireless local area network (WLAN) card or adapter. Additionally, or alternatively, the user device network interface 506 includes a radio frequency interface for wide area communications such as Long-Term Evolution (LTE) networks, or any other network now known or in the future developed. In some embodiments, the user device network interface 506 includes address, control, and/or data connections to enable appropriate communications on the network.

The user device memory 510 includes any non-transitory memory elements comprising one or more of volatile memory elements (for example, random access memory (RAM), nonvolatile memory elements (for example, read-only memory “ROM”), and combinations thereof). Moreover, in some embodiments, the user device memory 510 incorporates electronic, magnetic, optical, and/or other types of storage media now known or in the future developed. Note that, in an embodiment, the user device memory 510 has a distributed architecture, where various components are situated remotely from one another but are accessed by the user device processor 508. The software in the user device memory 510 includes one or more software programs, each of which includes an ordered listing of executable instructions for implementing logical functions. The software in the user device memory 510 includes a suitable user device operating system 514 and one or more user device applications 516. The user device operating system 514 controls the execution of other computer programs, such as, the one or more user device applications 516, and provides scheduling, input-output control, file and data management, memory management, and communication control and related services. The one or more user device applications 516 are configured to implement the various processes, algorithms, methods, techniques described herein.

The user device memory 510 further includes a user device data storage 520 used to store data. In the exemplary embodiment of FIG. 5, the user device data storage 520 is located internal to the user device memory 510 of the user device 106. Additionally, or alternatively, the user device data storage 520 are located external to the user device 106 such as, for example, an external hard drive connected to the user device user interface 504 (not shown). In a further embodiment, the user device data storage 520 are located external and connected to the user device 106 through a network and accessed via the user device network interface 506 (not shown).

In operation, information for storage, such as the inputs to modify the recommendations, in the user device data storage 520 is entered via the user device user interface 504. Alternatively, information, for example, the one or more recommendations, for storage in the user device data storage 520 is received from the server 102 via the user device transceiver 502. In some embodiments, the data stored in the user device data storage 520 is further provided to the database 108 and/or the server 102 during or after the execution of the medical procedure for updating the reference data stored in the database 108 and/or the server 102.

The user device 106 includes the user device transceiver 502. The user device transceiver 502 incorporated within a user device transceiver antenna (not shown), enables wireless communication between the devices, for example, the server 102 and the database 108 of FIG. 1. It will be appreciated by those of ordinary skill in the art that the user device 106 includes a single user device transceiver 502 as shown, or alternatively separate transmitting and receiving components, for example but not limited to, a transmitter, a transmitting antenna, a receiver, and a receiving antenna and/or any combination thereof.

The description below discusses the functions and operations performed by the respective server 102, imaging device 104, and user device 106. Although the description below discusses the functions and operations performed by the respective server 102, imaging device 104, and user device 106, a person skilled in the art would appreciate that, in some embodiments, the functions and operations of the server 102, the imaging device 104, and the user device 106 are performed in a single device or in a distributed manner by two or more devices without limiting the scope of the claimed subject matter.

The method 600 begins at 602, with the at least one imaging device 104 obtaining the one or more data associated with the one or more medical items 202 present in the predefined area of the medical procedure room 200. In an embodiment, as shown in FIGS. 7 through 9, when the predefined area corresponds to the first predefined area 708 surrounding the auxiliary table 202-2, the surgical scissor 202-4, the surgical needle 202-5, the forceps 202-6, and the surgical tweezer 202-7, the imaging device 104 obtains data associated with the setup and/or usage and/or count of the auxiliary table 202-2, the surgical scissor 202-4, the surgical needle 202-5, the forceps 202-6, and the surgical tweezer 202-7. In another embodiment, as shown in FIGS. 10 through 11, when the predefined area corresponds to the second predefined area 1002 including the entire area of the medical procedure room 200, the imaging device 104 obtains the data associated with the usage and/or setup and/or count of each medical item 202 present in the medical procedure room 200.

In accordance with various embodiments, at 604, the imaging device 104 is configured to transmit the one or more data associated with the one or more medical items 202 present in the predefined area 708, 1002 of the medical procedure room 200. At 606, the server 102 (for example, the server processor 410) is configured to receive the one or more data associated with the one or more medical items 202 present in the predefined area 708, 1002 of the medical procedure room 200 from the imaging device 104. The data is received via the server transceiver 402 and is stored in the server data storage 420 of the server memory 412.

In accordance with various embodiments, at 608, the server 102 (for example, the server processor 410) is configured to identify an upcoming phase of the medical procedure that is to be performed in the medical procedure room 200. The upcoming phase of the medical procedure corresponds to a phase/stage of the medical procedure that is planned or scheduled to occur after an ongoing (currently in progress or being executed) phase of the medical procedure. In an exemplary embodiment, the upcoming phase is an immediate phase that occurs after the ongoing phase. For example, when an ongoing phase is a preoperative phase, then the upcoming phase will be an intraoperative phase. The upcoming phase can be identified using a plurality of techniques. In an exemplary embodiment, the server 102 (for example, the server processor 410) is configured to identify the upcoming phase of the medical procedure based on a user input. For example, the server processor 410 displays a list of phases associated with the medical procedure on the user device display 512 and identifies the upcoming phase based on a selection made on the user device 106 by the user (for example, a medical practitioner or medical procedure room personnel).

In yet another embodiment, the server 102 (for example, the server processor 410) is configured to identify the upcoming phase of the medical procedure based on the data associated with the usage of the one or more medical items 202 present in the predefined area 708, 1002 of the medical procedure room 200 during an ongoing phase. To this end, the server 102 (for example, via the server transceiver 402) receives data associated with the usage of the medical items 202 in the predefined area 708, 1002 of the medical procedure room 200 from the imaging device 104 during the ongoing phase. The server 102 then compares the data with one or more prestored reference data (interchangeably referred to as the reference data) associated with the usage of the one or more medical items in a plurality of phases of the one or more reference medical procedures (for example, the reference medical procedures 330, 332) performed prior to the medical procedure. The server 102 is further configured to identify, based on the comparison, an ongoing phase associated with the medical procedure in the medical procedure room 200. For example, the server 102 receives data indicating a time stamp of usage of the surgical scissor 202-4, an order of usage of the surgical scissor 202-4, and a duration of the usage of the surgical scissor 202-4 in the medical procedure room 200 during the ongoing phase and compare the received data with the one or more prestored reference data associated with the surgical scissor 202-4 in a plurality of phases of the one or more reference medical procedures 330, 332. The server 102 then identifies a second phase as the ongoing phase, for example, by comparing the data associated with the usage of the surgical scissor 202-4 in the medical procedure room 200 with the prestored reference data indicating similar usage of the surgical scissor 202-4 in the second phases of the one or more reference medical procedures.

The server 102 is further configured to identify the upcoming phase of the medical procedure based on the identified ongoing phase. To this end, the server 102 (for example, the server processor 410) obtains a list of phases arranged in a sequential order for the medical procedure from, for example, the server memory 412 or the database 108, and identifies the phase listed subsequent to the ongoing phase as the upcoming phase. For example, the server 102 identifies the upcoming phase as a third phase (not shown) subsequent to the second phase, upon determining the ongoing phase as the second phase. In some embodiments, the server 102 is configured to provide a notification associated with the ongoing and/or upcoming phases of the medical procedure to one or more other users associated with the one or more user devices 106.

At 610, the server 102 (for example, the server processor 410) is configured to obtain the one or more reference data for the plurality of reference medical items required for the upcoming phase of the medical procedure from the one or more reference medical procedures 330, 332 performed prior to the medical procedure, for example, via the server transceiver 402. The reference data corresponds to the identification, setup, usage, and count of the reference medical items 202 associated with a phase of the reference medical procedures 330, 332. In accordance with various embodiments, the reference data is associated with a phase identifier of the upcoming phase. For example, as shown in FIGS. 3A and 3B, the server 102 upon determining that the upcoming phase is a second phase, obtains reference data, such as, identifier 308, usage 310 (including percentile, duration, time stamp of usage), count 312, arrangement 316, for a plurality of reference medical items, such as, the surgical scissor, the forceps, or the various chargeable and/or disposable medical item associated with the phase identifier P2 of the second phase of the one or more reference medical procedures 330, 332. In some embodiments, the server 102 is configured to obtain the reference data that is associated with one or more of the medical practitioner identifier 340 identifying the medical practitioner, the medical procedure identifier 342 identifying the medical procedure, and the medical procedure room identifier 346 identifying the medical procedure room 200. In yet other embodiments, the server 102 is configured to obtain the reference data that is associated with one or more of the plurality of medical practitioner identifiers 340, the plurality of medical procedure identifiers 342, and the plurality of medical procedure room identifiers 346.

At 612, the server 102 (for example, the server processor 410) is configured to compare the one or more reference data for the plurality of reference medical items with the one or more received data associated with the one or more medical items 202 present in the medical procedure room 200. At 614, the server 102 (for example, the server processor 410) is configured to provide the one or more recommendations for optimizing the medical procedure based on the comparison. In an exemplary embodiment, the one or more recommendations include recommendations for a removal of a medical item of the one or more medical items 202 present in the predefined area 708, 1002 of the medical procedure room 200, an addition of a medical item in the predefined area 708, 1002 of the medical procedure room 200, a change in position of a medical item 202 of the one or more medical items 202 present in the predefined area 708, 1002 of the medical procedure room 200, a usage of a medical item 202 of the one or more medical items 202 present in the predefined area 708, 1002 of the medical procedure room 200, and a change in a number of units of a medical item 202 (for example, chargeable and/or disposable medical item) in the predefined area 708, 1002 of the medical procedure room 200.

To this end, the server 102 (for example, the server processor 410 or the machine learning module 422 of the server processor 410) is configured to determine an optimal reference data corresponding to each phase of a medical procedure for comparison with the received data associated with the one or more medical items 202 present in the medical procedure room 200. The optimal reference data corresponds to one or more of an identification, a setup, a usage, and a count of a plurality of reference medical items required for each phase of a medical procedure. The optimal reference data is determined by analyzing the one or more reference data associated with the one or more reference medical procedures 330, 332. In an exemplary embodiment, when the one or more reference medical procedures 330, 332 includes one reference medical procedure (for example, the reference medical procedure 330), the optimal reference data corresponds to the reference data associated with the one reference medical procedure. In another exemplary embodiment, when the one or more reference medical procedures 330, 332 includes two or more reference medical procedures (for example, the reference medical procedures 330, 332), the optimal reference data is obtained by analyzing the plurality of reference data associated with each of the two or more reference medical procedures.

In an embodiment, the optimal reference data for each phase of the medical procedure is determined based on a quantity (or a number) of instances of the reference data in the corresponding phases of the one or more reference medical procedures. For example, when a medical item is associated with N number of corresponding phases of the one or more reference medical procedures and N is greater than a predefined threshold number, the reference data associated with the medical item is considered as the optimal reference data. Similarly, when a specific setup and/or a usage and/or a count of a medical item is associated with N number of corresponding phases of the one or more reference medical procedures and N is greater than the predefined threshold number, the reference data associated with the specific setup and/or usage and/or count of the medical item is considered as the optimal reference data. In an exemplary embodiment, the threshold number is provided by a user (such as, the medical practitioner or the medical procedure room personnel) and is configured to be changed depending upon the preference of the user. In some embodiments, different threshold numbers are defined for different reference medical items.

In some embodiments, the machine learning module 422 is configured to determine the optimal reference data by analyzing the reference data associated with a specific phase of the one or more reference medical procedures 330, 332 and determining an optimal relationship between the reference data and one or more performance parameters (such as, health parameters of a patient, time taken for the medical procedure) associated with the corresponding phase. In such cases, the server 102 is configured to determine the performance parameters based on data obtained from one or more sensors in the medical procedure room 200. For example, the machine learning module 422 determines that the medical item 202, such as, the forceps 202-6, when associated with the second phase reduces the time taken to complete the medical procedure. Similarly, the machine learning module 422 determines that the medical item 202, such as, the forceps 202-6, when used in a certain way results in achieving desired health parameters of a patient during the second phase. In such cases, the machine learning module 422 associates the forceps 202-6 and the determined way of usage of the forceps 202-6 with the second phase of the medical procedure for obtaining the optimal reference data.

In accordance with various embodiments, the server 102 is configured to provide a recommendation for the removal of a medical item from the predefined area 708, 1002 of the medical procedure room 200 by identifying the plurality of medical items 202 present in the predefined area 708, 1002 of the medical procedure room 200 based on the one or more data obtained from the imaging device 104. The medical item 202 is uniquely identified by a medical item identifier 308 (interchangeably referred to as identifier 308). For each of the plurality of medical items 202 present in the predefined area 708, 1002 of the medical procedure room 200, the server 102 is configured to compare the one or more reference data (for example, the optimal reference data) with the medical item identifier 308 corresponding to the medical item 202 and determine whether the medical item 202 is required for the upcoming phase of the medical procedure based on the comparison. The server 102 then provides the recommendation for removing the medical item 202 from the predefined area 708, 1002 of the medical procedure room 200 for the upcoming phase when the medical item 202 is not required for the upcoming phase.

For example, when the upcoming phase is a second phase and the server 102 identifies a presence of the surgical needle 202-5 in the predefined area 708, 1002 of the medical procedure room 200, the server 102 compares an identifier of the surgical needle 202-5 with the identifiers 308 of the plurality of reference medical items 202 required for the second phase (the optimal reference data). When the plurality of reference medical items 202 required for the second phase does not indicate the requirement of the surgical needle 202-5, the server 102 is configured to provide the recommendation for removing the surgical needle 202-5 from the predefined area 708, 1002 of the medical procedure room 200 for the second phase.

In accordance with various embodiments, the server 102 is configured to provide a recommendation for addition of a medical item in the predefined area 708, 1002 of the medical procedure room 200 by identifying the plurality of medical items 202 present in the predefined area 708, 1002 of the medical procedure room 200 based on the one or more data obtained from the imaging device 104. Each medical item 202 is uniquely identified by a medical item identifier 308. The server 102 is further configured to compare the one or more reference data (for example, the optimal reference data) with the medical item identifiers 308 corresponding to the one or more medical items 202 present in the predefined area 708, 1002 and determine an absence of the medical item 202 in the predefined area 708, 1002 of the medical procedure room 200 for the upcoming phase of the medical procedure based on the comparison. The server 102 further provides the recommendation for adding the medical item 202 in the predefined area 708, 1002 of the medical procedure room 200 for the upcoming phase based on the determination.

For example, when the upcoming phase is the second phase and the server 102 identifies a presence of the surgical needle 202-5, the forceps 202-6, the surgical scissor 202-4 in the predefined area 708, 1002 of the medical procedure room 200, the server 102 compares the identifiers of the surgical needle 202-5, the forceps 202-6, the surgical scissor 202-4 with the identifiers 308 of the plurality of reference medical items 202 required for the second phase (for example, the optimal reference data) to determine whether all medical items 202 required for the second phase are present in the predefined area 708, 1002 of the medical procedure room 200. When the plurality of reference medical items 202 required for the second phase indicates one or more medical items 202 that are not present in the predefined area 708, 1002 of the medical procedure room 200 but are required for the second phase, the server 102 recommends the addition of the one or more medical items 202.

In accordance with various embodiments, the server 102 is configured to provide a recommendation for the change in an arrangement of a medical item of the one or more medical items 202 present in the predefined area 708, 1002 of the medical procedure room 200. To recommend a change in the arrangement of the medical item 202, the server 102 is configured to identify one or more data associated with the arrangement of the medical item 202 present in the predefined area 708, 1002 of the medical procedure room 200. The server 102 is configured to compare the one or more data associated with the arrangement of the medical item 202 with the arrangement of the plurality of reference medical items (for example, the optimal reference data) associated with the identifier 308 of the medical item 202. The server 102 then determines a change in the arrangement of the medical item 202 present in the predefined area 708, 1002 of the medical procedure room 200 based on the comparison. The server 102 is further configured to provide the recommendation for changing the arrangement of the medical item 202 present in the predefined area 708, 1002 of the medical procedure room 200 for the upcoming phase based on the determination.

In accordance with various embodiments, the one or more recommendations include a recommendation associated with the usage of a medical item 202 present in the predefined area 708, 1002 of the medical procedure room 200. The server 102 is configured to receive data associated with the usage of a medical item 202 in the predefined area 708, 1002 of the medical procedure room 200, from the imaging device 104. The medical item 202 is associated with a medical item identifier 308. The server 102 is further configured to compare the data associated with the usage of the medical item 202 associated with the medical item identifier 308 with one or more reference data (for example, the optimal reference data) corresponding to reference data associated with the usage of a reference medical item associated with the medical item identifier from the one or more reference medical procedures 330, 332. The server 102 then determines a deviation in the data associated with the usage of the medical item 202 with respect to the reference data associated with the usage 310 of the reference medical item 202 and provides the recommendation associated with the usage of the medical item 202 based on the deviation.

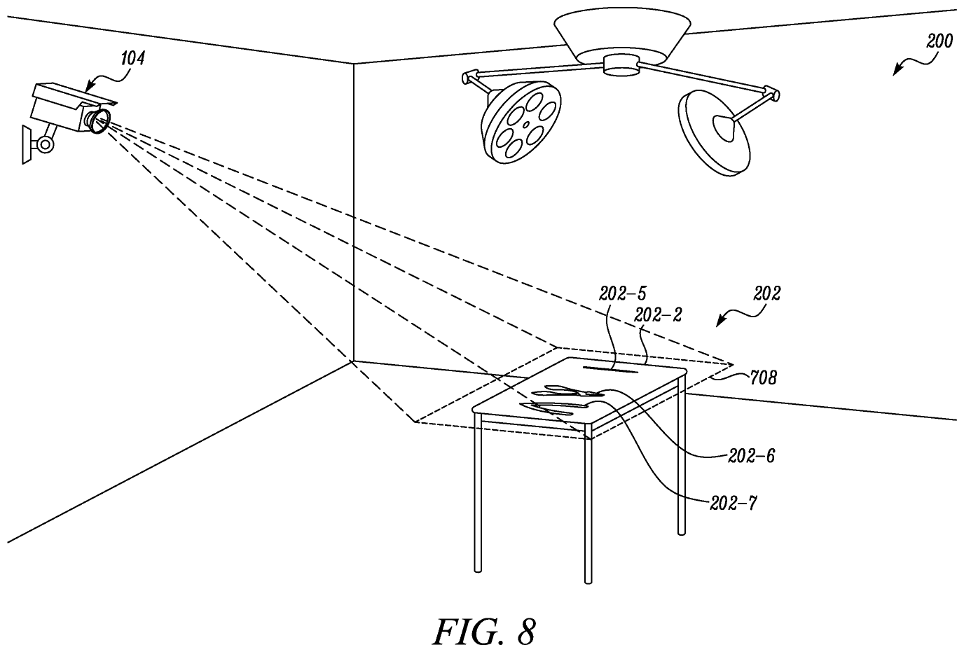

The server 102 obtains the one or more data related to the usage of the medical items 202, for example, the surgical scissor 202-4, in the predefined area 704, 1002 of the medical procedure room 200 using various techniques. In an embodiment, as shown in FIGS. 7 through 9, the data related to the usage of the surgical scissor 202-4 is obtained based on the presence or removal of the surgical scissor 202-4 from the first predefined area 708 of the medical procedure room 200. For example, as shown in FIG. 7, the imaging device 104 obtains data that indicates that the surgical scissor 202-4 is placed on the auxiliary table 202-2 of the medical procedure room 200. The imaging device 104 continuously obtains the data associated with the arrangement of the surgical scissor 202-4 and obtains data (such as, a time stamp) associated with the removal of the surgical scissor 202-4 from the auxiliary table 202-2 (as shown in FIG. 8). The imaging device 104 further obtains the data (such as, a time stamp) associated with the placement of the surgical scissor 202-4 back on the auxiliary table 202-2. The data obtained by the imaging capturing device 104 is utilized by the server 102 to determine the data associated with the usage, such as, the time stamp of the usage of the surgical scissor 202-4, the duration of the usage of the surgical scissor 202-4, and the order of the usage of the surgical scissor 202-4 for an ongoing phase of the medical procedure.

In yet another embodiment, as shown in FIGS. 10 through 11, the data related to the usage of the surgical scissor 202-4 is obtained based on the movement of the surgical scissor 202-4 in the second predefined area 1002 of the medical procedure room 200. For example, as shown in FIG. 10, the imaging device 104 obtains data that indicates that the surgical scissor 202-4 is placed on the auxiliary table 202-2 of the medical procedure room 200. The imaging device 104 continuously obtains the data associated with the surgical scissor 202-4 and obtains position data associated with the movement of the surgical scissor 202-4 in the predefined area 1002 (as shown in FIG. 11). The data obtained by the imaging device 104 is utilized by the server 102 to determine the data associated with the usage, such as, a time stamp of the usage of the surgical scissor 202-4 and a duration of the usage of the surgical scissor 202-4 for an ongoing phase of the medical procedure in the medical procedure room 200.

In accordance with various embodiments, the server 102 compares the obtained data with the data associated with the usage of surgical scissor 202-4 in the one or more reference medical procedures 330, 332. The server 102 further determines a deviation between the obtained data and the data associated with the one or more reference medical procedures 330, 332, such as, a deviation in the duration of use of the surgical scissor 202-4 between the obtained data and the data associated with the one or more reference medical procedures 330, 332, and provides the recommendation associated with the duration of usage of the surgical scissor 202-4.

In accordance with various embodiments, the server 102 is configured to provide a recommendation for the change in a number of units of a medical item 202 (for example, the chargeable and/or disposable medical items) in the predefined area 708, 1002 of the medical procedure room 200 by identifying the number of units of each medical item 202 present in the predefined area 708, 1002 of the medical procedure room 200 based on the one or more data obtained from the imaging device 104. Each unit of the medical item 202 is identified by a same medical item identifier 308 (interchangeably referred to as identifier 308). For each medical item 202 present in the predefined area 708, 1002 of the medical procedure room 200, the server 102 is configured to compare the one or more reference data (for example, the optimal reference data) with the medical item identifier 308 corresponding to the medical item 202 and determine the count 312 of the medical item 202 that represents the number of units of the medical item 202 that were previously utilized or consumed during the upcoming phase of the one or more reference medical procedures based on the comparison. The server 102 then provides the recommendation for modifying or changing the number of units of the medical item 202 in the predefined area 708, 1002 of the medical procedure room 200 for the upcoming phase when the number of units of the medical item 202 present in the medical procedure room 200 does not match the count 312 in the one or more reference data. In some embodiments, the server 102 is configured to determine a total count of units of a medical item 202 required during the medical procedure based on a summation of the number of units of the medical item 202 required during each phase of the medical procedure. In such cases, the server 102 compares the total count with the number of units of the medical item 202 present in the medical procedure room 200 to provide recommendations.

For example, in most medical procedures, the packaging of a number of units of a medical item 202 (for example, the chargeable and/or disposable medical items) is removed and all such units of the medical item 202 are laid out, for example, on the auxiliary table 202-2, in the medical procedure room 200 before the start of the medical procedure or a particular phase of the medical procedure. In many cases, not all units of such chargeable and/or disposable medical items 202 are utilized during the medical procedure or the particular phase of the medical procedure, as a result of which the unused units of the chargeable and/or disposable medical items are required to be discarded at the end of the medical procedure. In such cases, in order to reduce the wastage associated with the chargeable and/or disposable medical items 202, the server 102 is configured to provide recommendations on the user device 106, before the start of the medical procedure or the particular phase of the medical procedure, to modify the number of units of the medical item 202 in the medical procedure room 200 to match the count 312 or the total count (described above) so that the packaging of only the required number of units of the medical item 202 is removed or opened.

In accordance with various embodiments, the server 102 is configured to provide the one or more recommendations during a predefined period prior to the start of the upcoming phase. In some embodiments, the server 102 is also configured to continuously obtain the data associated with the medical items 202 present in the predefined area of the medical procedure room 200 during the execution of the upcoming phase (for example, when the upcoming phase becomes the ongoing phase) and provides real-time recommendations to optimize the set-up and/or usage and/or count of the medical items 202 in the medical procedure room 200. In some additional embodiments, the server 102 is configured to provide the recommendations after the completion of the medical procedure as feedback to the medical practitioner. The server 102 is configured to provide the recommendations and the obtained one or more data associated with the medical items 202 in the medical procedure room 200 to the database 108 to update the one or more reference data. The database 108 stores the received recommendations and the one or more data associated with the medical items 202 as a reference data for providing recommendations for the next medical procedure(s).

In accordance with various embodiments, the server 102 is configured to continuously optimize the medical procedure, using one or more machine learning algorithms of the machine learning module 422. To this end, the server 102 is configured to continuously monitor the data associated with the one or more medical items 202 in the medical procedure room 200 and update the optimal relationship between the reference data and the one or more performance parameters (such as, health parameters of a patient, time taken for the medical procedure) defined by the user. The machine learning module 422 is then configured to update the one or more recommendations, for example, in real-time, based on the updated optimal relationship. Alternatively, in some embodiments, the updated recommendations are provided before the start of a phase or after an end of the phase of the medical procedure.

In accordance with various embodiments, at 616, the user device 106 (for example, the user device processor 508) is configured to receive the one or more recommendations from the server 102 via the user device transceiver 502 and display (for example, on the user device display 512) the received one or more recommendations for the optimization of the medical procedure at 618. For example, the one or more recommendations are displayed on the augmented reality device 106-n. In some embodiments, the user device 106 is configured to receive the inputs to modify the recommendations displayed on the user device display 512. For example, the inputs correspond to changing the setup and/or usage and/or count of the medical item 202 for a particular phase of the medical procedure. The inputs for modifying the recommendations depend on one or more of the preference of the medical practitioner, the layout of the medical procedure room, and the type of the medical procedure. The inputs can be provided at any time, for example, before, during, and/or after a particular phase of the medical procedure. The recommendations and inputs to modify the recommendations are further provided to the server 102 and/or the database 108, via the user device transceiver 502, to update the one or more reference data.

In one embodiment, displaying the one or more recommendations on the augmented reality device 106-n includes displaying a virtual representation of a medical item 202. In accordance with various embodiments, the virtual representation of a medical item 202 is used to provide recommendations associated with the addition of the medical item 202, the usage of the medical item 202, the change (for example, an increase) in a number of units of the medical item 202, and/or an optimal arrangement of the medical item 202 in the predefined area 708, 1002 of the medical procedure room 200. For example, a recommendation associated with the addition of the medical item 202, such as, a surgical clamp, is provided by displaying a virtual representation of the surgical clamp on the augmented reality device 106-n. In accordance with various embodiments, the virtual representation of the medical item 202 is displayed at a location in the predefined area 708, 1002 of the medical procedure room 200. For example, when the server 102 determines that the surgical clamp needs to be placed on the auxiliary table 202-2 in the medical procedure room 200 for the upcoming phase, the virtual representation of the surgical clamp is displayed on the auxiliary table 202-2 in the virtual reality view to guide the user with the correct placement. In some embodiments, displaying the one or more recommendations on the augmented reality view of the user device 106 includes displaying a color-coded indication on the virtual image of a medical item 202 present in the predefined area 708, 1002 of the medical procedure room 200

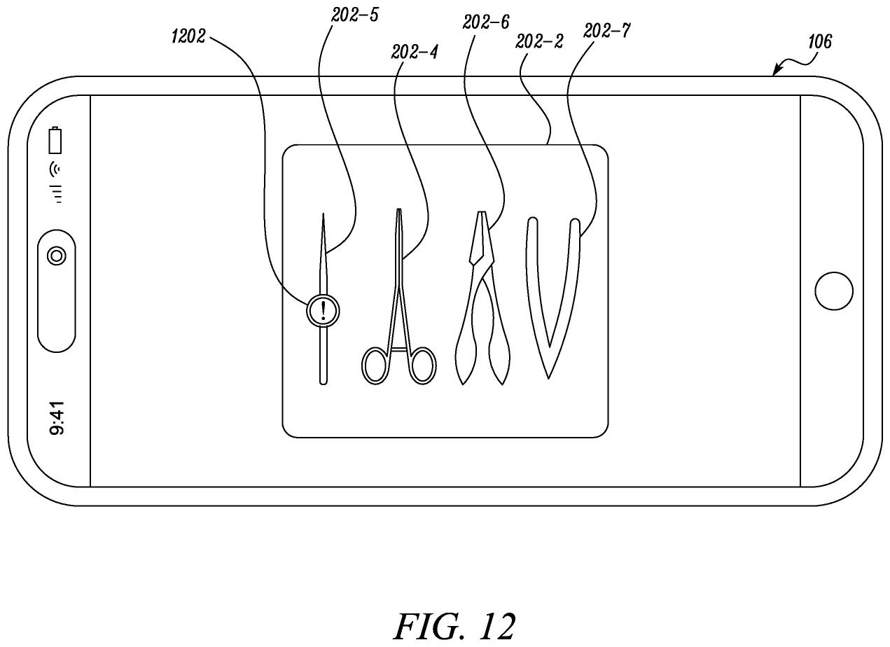

In some embodiments, displaying the one or more recommendations on the augmented reality view of the user device 106 includes displaying superimposition of a digital identification mark on a medical item 202 present in the predefined area 708, 1002 of the medical procedure room 200. In accordance with various embodiments, the superimposition of a digital identification mark is used to provide recommendations associated with the removal of the medical item 202 from the predefined area 708, 1002 of the medical procedure room 200 or the change (for example, a decrease) in a number of units of the medical item 202. For example, as shown in FIG. 12, a recommendation associated with the removal of the medical item 202, such as, the surgical needle 202-5, is provided by displaying a digital identification mark 1202 on the augmented reality view of the surgical needle 202-5 on the user device 106.

In some embodiments, as shown in FIGS. 6A and 6B, all steps performed by the imaging device 104, the server 102 and the user device 106, for example, but not limited to, receiving the one or more data associated with the medical items 202, obtaining the one or more reference data, comparing the obtained one or more data with the one or more reference data, and providing recommendations are repeated after a predefined interval to provide real-time recommendations for optimizing each phase of the medical procedure.

In some embodiments, all steps performed by the imaging device 104, the server 102 and the user device 106, for example, but not limited to, receiving the one or more data associated with the medical items 202, obtaining the one or more reference data, comparing the obtained one or more data with the one or more reference data, and providing recommendations are performed during a predefined period prior to a start of the upcoming phase. In accordance with various embodiments, the predefined period is defined using the user device 106 or the server 102 based on, for example, a time required to setup the medical items 202 for the upcoming phase.

Referring to FIG. 13, the optimization cycle 1300 associated with the optimizing the medical procedure is described. At 1302, the server 102 (for example, the server processor 410) obtains the one or more reference data for the plurality of reference medical items 202 required for the upcoming phase of the medical procedure from the one or more reference medical procedures performed prior to the medical procedure, via the server transceiver 402. At 1304, the server 102 (for example, the server processor 410) receives the one or more data associated with the one or more medical items 202 present in the predefined area 708, 1002 of the medical procedure room 200 from the imaging device 104 via the server transceiver 402. At 1306, the server 102 (for example, the server processor 410) compares the one or more reference data for the plurality of reference medical items with the one or more received data associated with the one or more medical items 202 present in the medical procedure room 200 and provides the one or more recommendations at 1308. The recommendations provided by the server 102 are then utilized to update the one or more reference data. In some embodiments, the one or more data associated with the medical items 202 in the medical procedure room 200 is also utilized to update the one or more reference data. The updated one or more reference data is then used to provide recommendations for the next medical procedure(s).

The system and the method of the present disclosure are directed towards optimizing a medical procedure by providing recommendations to the user, such as, a medical practitioner or a medical procedure room personnel, to achieve an optimal setup and/or usage and/or count of the medical items present in the medical procedure room 200. In the present disclosure, the medical procedure is divided into various phases and the medical procedure is optimized by optimizing the usage and/or setup and/or count of medical items in each phase. Phase-wise optimization of the medical procedure provides various advantages like improvement in the processing speed of the server 102 as the server 102 processes and compares a subset of the reference data (for example, the reference data associated with a few medical items of the corresponding phase only) and not a reference data of all medical items required during the entire medical procedure to achieve the optimization. Also, the phase wise optimization approach provides more accurate setup and/or usage and/or count optimization of each medical item, especially in a scenario when a same medical item is required to be utilized or arrangement differently for different phases of the medical procedure. In such cases, by providing the data related to optimization at each individual phase level, the accuracy of the optimization can be improved further. Moreover, along with providing recommendations for the upcoming phase, the system and method of the present invention provides real time feedback to the medical practitioners or medical procedure room personnels that can be used for further optimizing the medical procedure by eliminating any chances of error. Additionally, the dedicated setup of the medical items corresponding to the phase helps attain better clarity about the ongoing and upcoming phases of the medical procedures and reduces the unwanted delays.

Furthermore, the identification of an ongoing and/or upcoming phase of the medical procedure enables the user, for example, the medical practitioner and the medical procedure room personnel, to notify the progression of the medical procedure to one or more other users associated with the medical procedure. This is especially helpful, when coordination between multiple medical teams is required to complete the medical procedure. The notification related to the ongoing and/or the upcoming phase of the medical procedure enables better coordination between the multiple medical teams.

In the foregoing specification, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings.

The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

Moreover, in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element preceded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The term “coupled” as used herein is defined as connected, although not necessarily directly and not necessarily mechanically. A device or structure that is “configured” in a certain way is configured in at least that way but may also be configured in ways that are not listed.

It will be appreciated that some embodiments may be comprised of one or more generic or specialized processors (or “processing devices”) such as microprocessors, digital signal processors, customized processors and field programmable gate arrays (FPGAs) and unique stored program instructions (including both software and firmware) that control the one or more processors to implement, in conjunction with certain non-processor circuits, some, most, or all of the functions of the method and/or apparatus described herein. Alternatively, some or all functions could be implemented by a state machine that has no stored program instructions, or in one or more application specific integrated circuits (ASICs), in which each function or some combinations of certain of the functions are implemented as custom logic. Of course, a combination of the two approaches could be used.

Moreover, an embodiment can be implemented as a computer-readable storage medium having computer readable code stored thereon for programming a computer (for example, comprising a processor) to perform a method as described and claimed herein. Examples of such computer-readable storage mediums include, but are not limited to, a hard disk, a CD-ROM, an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory) and a Flash memory. Further, it is expected that one of ordinary skill, notwithstanding possibly significant effort and many design choices motivated by, for example, available time, current technology, and economic considerations, when guided by the concepts and principles disclosed herein will be readily capable of generating such software instructions and programs and ICs with minimal experimentation.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

1. A system for optimizing a medical procedure in a medical procedure room, the system comprising:
an imaging device configured to:
obtain one or more data associated with one or more medical items present in a predefined area of the medical procedure room;
a server communicatively coupled to the imaging device, the server configured to:
receive the one or more data associated with the one or more medical items present in the predefined area of the medical procedure room from the imaging device;
identify an upcoming phase of the medical procedure, wherein each medical procedure is divided into a plurality of phases, each phase uniquely identified by a phase identifier;
obtain one or more reference data for a plurality of reference medical items required for the upcoming phase of the medical procedure from one or more reference medical procedures performed prior to the medical procedure, wherein the one or more reference data for the plurality of reference medical items is associated with a phase identifier of the upcoming phase;
compare the one or more reference data for the plurality of reference medical items with the one or more received data associated with the one or more medical items present in the medical procedure room; and
provide one or more recommendations for optimizing the medical procedure based on the comparison; and
an augmented reality device communicatively coupled to the server, the augmented reality device configured to:
receive the one or more recommendations from the server; and
display the one or more recommendations on the augmented reality device for the optimization of the medical procedure.

2. The system of claim 1, wherein the one or more reference data includes identification of the plurality of reference medical items required for the upcoming phase, wherein the server is further configured to provide a recommendation of the one or more recommendations including removal of a medical item of the one or more medical items present in the predefined area of the medical procedure room, and wherein to remove the medical item, the server is configured to:
identify the medical item present in the predefined area of the medical procedure room based on the one or more data obtained from the imaging device, the medical item uniquely identified by a medical item identifier;
compare the reference data with the medical item identifier corresponding to the medical item;
determine whether the medical item is required for the upcoming phase of the medical procedure based on the comparison; and
provide the recommendation for removing the medical item from the predefined area of the medical procedure room for the upcoming phase when the medical item is not required for the upcoming phase.

3. The system of claim 1, wherein the one or more reference data includes identification of the plurality of reference medical items required for the upcoming phase, wherein the server is configured to provide a recommendation of the one or more recommendations including addition of a medical item in the predefined area of the medical procedure room, and wherein to add the medical item, the server is configured to:
identify the one or more medical items present in the predefined area of the medical procedure room based on the one or more data obtained from the imaging device, each medical item uniquely identified by a medical item identifier;
compare the one or more reference data with the medical item identifiers corresponding to the one or more medical items present in the predefined area;
determine an absence of the medical item in the predefined area of the medical procedure room for the upcoming phase of the medical procedure based on the comparison; and
provide the recommendation for adding the medical item in the predefined area of the medical procedure room for the upcoming phase based on the determination.

4. The system of claim 1, wherein the one or more reference data includes arrangement information of the plurality of reference medical items required for the upcoming phase, wherein the server is further configured to provide a recommendation of the one or more recommendations including change in an arrangement of a medical item of the one or more medical items present in the predefined area of the medical procedure room, and wherein to recommend change in the arrangement of the medical item, the server is configured to:
identify one or more data associated with the arrangement of the medical item present in the predefined area of the medical procedure room;
compare the one or more data associated with the arrangement of the medical item with the position information of the plurality of reference medical items;
determine a change in the arrangement of the medical item present in the predefined area of the medical procedure room based on the comparison; and
provide the recommendation for changing the arrangement of the medical item present in the predefined area of the medical procedure room for the upcoming phase based on the determination.

5. The system of claim 1, wherein to identify the upcoming phase of the medical procedure, the server is further configured to:
receive data associated with usage of the one or more medical items in the predefined area of the medical procedure room from the imaging device;
compare the data associated with the usage of the one or more medical items with one or more prestored reference data associated with the usage of the one or more medical items in a plurality of phases of the one or more reference medical procedures performed prior to the medical procedure;
identify, based on the comparison, an ongoing phase associated with the medical procedure in the medical procedure room; and
determine the upcoming phase of the medical procedure with reference to the ongoing phase.

6. The system of claim 1, wherein the server is further configured to identify the upcoming phase of the medical procedure based on a user input.

7. The system of claim 1, wherein the one or more reference data is associated with one or more of a medical practitioner identifier identifying the medical practitioner performing the medical procedure, a medical procedure identifier identifying the medical procedure, and a medical procedure room identifier identifying the medical procedure room.

8. The system of claim 1, wherein the steps of receiving, identifying, obtaining, comparing, and providing are performed during a predefined period prior to a start of the upcoming phase.

9. The system of claim 1, wherein the server is further configured to provide a recommendation of the one or more recommendations including providing one or more data associated with a usage of a medical item in the predefined area of the medical procedure room, wherein to provide the one or more data, the server is configured to:
receive data associated with a usage of a medical item of the one or more medical items in the predefined area of the medical procedure room from the imaging device, wherein the medical item is associated with a medical item identifier;
compare the data associated with the usage of the medical item associated with the medical item identifier with one or more prestored reference data corresponding to reference data associated with a usage of a reference medical item associated with the medical item identifier from the one or more reference medical procedures performed prior to the medical procedure;
determine a deviation in the data associated with the usage of the medical item with respect to the reference data associated with the usage of the reference medical item; and
provide the recommendation associated with the usage of the medical item based on the deviation.

10. The system of claim 1, wherein displaying the one or more recommendations on the augmented reality device includes displaying a virtual representation of a medical item at a location in the predefined area of the medical procedure room.

11. The system of claim 1, wherein displaying the one or more recommendations on the augmented reality device includes displaying superimposition of a digital identification mark on a medical item present in the predefined area of the medical procedure room.

12. The system of claim 1, wherein the server is configured to compare the one or more reference data for the plurality of reference medical items with the one or more received data associated with the one or more medical items present in the medical procedure room by:
analyzing the one or more reference data associated with the one or more reference medical procedures;
determining an optimal reference data corresponding to each phase of the medical procedure based on the analyzed one or more reference data, the optimal reference data corresponding to one or more of an identification, a setup, and a usage of the plurality of reference medical items required for each phase of the medical procedure, wherein the optimal reference data is determined by comparing a number of instances of the one or more reference data in the corresponding phases of the one or more reference medical procedures with a predefined threshold number; and
comparing the received one or more data associated with the one or more medical items with the optimal reference data to provide recommendations.

13. A method for optimizing a medical procedure in a medical procedure room, the method comprising:
obtaining, by at least one imaging device, one or more data associated with one or more medical items present in a predefined area of the medical procedure room;
transmitting, by the at least one imaging device, the one or more data associated with the one or more medical items present in the predefined area of the medical procedure room;
receiving, by a server, the one or more data associated with the one or more medical items present in the predefined area of the medical procedure room from the at least one imaging device;
identifying, by the server, an upcoming phase of the medical procedure, wherein each medical procedure is divided into a plurality of phases, each phase uniquely identified by a phase identifier;
obtaining, by the server, one or more reference data for a plurality of reference medical items required for the upcoming phase of the medical procedure from one or more reference medical procedures performed prior to the medical procedure, wherein the one or more reference data for the plurality of reference medical items is associated with a phase identifier of the upcoming phase;
comparing, by the server, the one or more reference data for the plurality of reference medical items with the received one or more data associated with the one or more medical items present in the medical procedure room;
providing, by the server, one or more recommendations for optimizing the medical procedure based on the comparison;
receiving, by an augmented reality device, the one or more recommendations from the server; and
displaying, on the augmented reality device, the one or more recommendations for the optimization of the medical procedure.

14. The method of claim 13, wherein the one or more reference data includes an identification of the plurality of reference medical items required for the upcoming phase, wherein providing a recommendation of the one or more recommendations includes removal of a medical item of the one or more medical items present in the predefined area of the medical procedure room, and wherein the removal of the medical item includes:
identifying, by the server, the medical item present in the predefined area of the medical procedure room based on the one or more data obtained from the imaging device, the medical item uniquely identified by a medical item identifier;
comparing, by the server, the one or more reference data with the medical item identifier corresponding to the medical item;
determining, by the server, the medical item is not required for the upcoming phase of the medical procedure based on the comparison; and
providing, by the server, the recommendation for removing the medical item from the predefined area of the medical procedure room for the upcoming phase when the medical item is not required for the upcoming phase.

15. The method of claim 13, wherein the one or more reference data includes identification of the plurality of reference medical items required for the upcoming phase, wherein providing a recommendation of the one or more recommendations includes addition of a medical item in the predefined area of the medical procedure room, and wherein the addition of the medical item includes:
identifying, by the server, the one or more medical items present in the predefined area of the medical procedure room based on the one or more data obtained from the imaging device, each medical item uniquely identified by a medical item identifier;
comparing, by the server, the one or more reference data with the medical item identifiers corresponding to the one or more medical items present in the predefined area;
determining, by the server, an absence of the medical item in the predefined area of the medical procedure room for the upcoming phase of the medical procedure based on the comparison; and
providing, by the server, the recommendation for adding the medical item in the predefined area of the medical procedure room for the upcoming phase based on the determination.

16. The method of claim 13, wherein the one or more reference data includes position information of the plurality of reference medical items required for the upcoming phase, wherein providing a recommendation of the one or more recommendations includes change in an arrangement of a medical item of the one or more medical items present in the predefined area of the medical procedure room, and wherein recommending the change in the arrangement of the medical item includes:
identifying, by the server, one or more data associated with arrangement of the medical item present in the predefined area of the medical procedure room;
comparing, by the server, the one or more data associated with the arrangement of the medical item with the position information of the plurality of reference medical items;
determining, by the server, a change in the arrangement of the medical item present in the predefined area of the medical procedure room based on the comparison; and
providing, by the server, the recommendation for changing the arrangement of the medical item present in the predefined area of the medical procedure room for the upcoming phase based on the determination.

17. The method of claim 13, wherein identification of the upcoming phase of the medical procedure includes:
receiving, by the server, data associated with usage of the one or more medical items in the predefined area of the medical procedure room from the imaging device;
comparing, by the server, data associated with the usage of the one or more medical items with a prestored one or more reference data associated with the usage of the one or more medical items in a plurality of phases of the one or more reference medical procedures performed prior to the medical procedure;
identifying, by the server, based on the comparison, an ongoing phase associated with the medical procedure in the medical procedure room; and
determining, by the server, the upcoming phase of the medical procedure with reference to the ongoing phase.

18. The method of claim 13, wherein the steps of obtaining, transmitting, receiving, identifying, obtaining, comparing, providing, receiving, and displaying are performed during a predefined period prior to a start of the upcoming phase.

19. The method of claim 13, wherein providing a recommendation of the one or more recommendations includes providing one or more data associated with a usage of a medical item in the predefined area of the medical procedure room, wherein providing the one or more data includes:
receiving, by the server, data associated with a usage of a medical item of the one or more medical items in the predefined area of the medical procedure room from the imaging device, wherein the medical item is associated with a medical item identifier;
comparing, by the server, the data associated with the usage of the medical item associated with the medical item identifier with a prestored one or more reference data corresponding to reference data associated with a usage of a reference medical item associated with the medical item identifier from the one or more reference medical procedures performed prior to the medical procedure;
determining, by the server, a deviation in the data associated with the usage of the medical item with respect to the reference data associated with the usage of the reference medical item; and
providing, by the server, the recommendation associated with the usage of the medical item based on the deviation.

20. The method of claim 13, wherein comparing the one or more reference data for the plurality of reference medical items with the one or more received data associated with the one or more medical items present in the medical procedure room includes:
analyzing the one or more reference data associated with the one or more reference medical procedures;
determining an optimal reference data corresponding to each phase of the medical procedure based on the analyzed one or more reference data, the optimal reference data corresponding to one or more of an identification, a setup, and a usage of the plurality of reference medical items required for each phase of the medical procedure, wherein the optimal reference data is determined by comparing a number of instances of the one or more reference data in the corresponding phases of the one or more reference medical procedures with a predefined threshold number; and
comparing the received one or more data associated with the one or more medical items with the optimal reference data to provide recommendations.
